(19) United States
(12) Patent Application Publication
(10) Pub. No.: US 2006/0062863 A1
(43) Pub. Date: Mar. 23, 2006

(54) Compositions for anti-obesity, health-restorative and health-promotional benefits

(76) Inventor: Shibnath Ghosal, Calcutta (IN)

Correspondence Address: MATHEWS, SHEPHERD, MCKAY, & BRUNEAU, P.A., 100 THANET CIRCLE, SUITE 306, PRINCETON, NJ, 08540, US

(21) Appl. No.: 11/229,871

(22) Filed: Sep. 19, 2005

Related U.S. Application Data
(60) Provisional application No. 60/612,106, filed on Sep. 22, 2004.

(51) Int. Cl. A61K 36/48

(52) U.S. Cl. 424/757

(57) ABSTRACT

An obesity control agent with health-restorative and health-promotional benefits to humans comprising the extract of Chlorophytum species, more particularly, Chlorophytum arundinacceum, is disclosed. The bioactive principles responsible for anti-obesity property have been determined to be mainly due to spirosta-steroidal saponins, spirosta-steroidal alkaloids and galacto-glucan oligosaccharides. Most effective as an obesity control agent is the spirosta-steroidal saponins. Pharmaceutical, nutritional and veterinary use of this inventive composition is also disclosed.

### CROSS REFERENCE TO RELATED APPLICATION

This application claims the benefit of U.S. Provisional Patent Application No. 60/612,106, filed Sep. 22, 2004, the entirety of which is hereby incorporated by reference into this application.

### BACKGROUND OF THE INVENTION

1. Field of the Invention

This invention relates to compositions for anti-obesity, health-restorative and health promotional benefits to mammals, more particularly humans. This composition is obtained from higher plants belonging to genus Chlorophytum. The extraction process for obtaining such composition, as well as pharmaceutical, nutritional and veterinary use products thereof, are also disclosed.

2. Description of the Related Art

Chlorophytum arundinaceum Baker (family—Liliaceae) (Hindi, Safed Musli) is a reputed Indian medicinal plant, whose root-tubers are used as an aphrodisiac and for health-restorative and health-promotional purposes in Ayurvedic medicine since the 2^{nd} Century B.C. See for example, Sharma, P. V. (1978), Dravyaguna Vijnan, p. 559. Chaukhamba Sanskrit Sansthan, Varanasi, India. More than 175 species of Chlorophytum have been reported in the world. Chlorophytum comosum is widely used as ornamental plant and is commonly known as spider ivy, spider plant, aeroplane plant, or walking anthericum. In India, about eight species under the name of Safed Musli are reported. These are as follows:
1. Chlorophytum arundinaceum
2. Chlorophytum borivilianum
3. Chlorophytum tuberosum
4. Chlorophytum malabericum
5. Chlorophytum attenuatum
6. Chlorophytum breviscapum
7. Asparagus filicinus
8. Asparagus gonoclados

Out of these (listed above), only Chlorophytum arundinaceum, Chlorophytum borivilianum and Chlorophytum tuberosum are usually collected for commercial purposes from the wild. Chlorophytum borivilianum Sant. & F. is reported in Bastar Forests (Madhya Pradesh), Dangs forest (Gujarat), Mount Abu, Mahi, Aravalli hills (Rajasthan) of India. It is also reported to occur in some parts of Pakistan.

Chlorophytum arundinaceum is reported to be available in many parts of India: all districts of Chota Nagpur, Vindhya, Satpura & Aravali Hills, parts of central India, Taria region of North-East Himalayas in Assam, West Bengal, and Bihar states. Chlorophytum is found in soils rich in organic matter. It requires bright sunlight for growth. It is now widely cultivated in different parts of India like Andhra Pradesh, Rajashthan, Gujarat, Maharastra on commercial basis. The crop is a popular rainy season crop in India and a commercial root harvest can be obtained in 3-4 months.

The beneficial effects attributed to this plant in Ayurveda are for Chlorophytum arundinaceum Baker (Liliaceae), and not Chlorophytum borivilianum. In fact, this species can be regarded as a substitute for Asparagus adscendens Roxb. (Liliaceae). It is the Asparagus adscendens which is originally known as Safed Musli in Ayurvedic literature Extracts of both these plants (Chlorophytum arundinaceum and Chlorophytum borivilianum) are used in herbal medicines as aphrodisiac and tonic (vitalizer). The chemical constituents of both, which are mainly responsible for these bioactivities, constitute steroidal spiroketal saponins, their genins and glycoalkaloids. It should be noted that Panax ginseng does not contain spiroketal steroidal saponins/sapogenins, but only triterpenoidal saponins. The bioactivities of spiroketal steroidal saponins/sapogenins would considerably differ from those of triterpenoidal saponins. Moreover, Panax ginseng saponins are known to suffer from several adverse side-effects, not present in Safed Musli saponins.

Obesity is known to impair libido and can also adversely affect health due to adverse systemic signaling by the deposited fat, however, no scientific study is reported that assesses the anti-obesity and immuno-modulatory effects of Cholorphytum species.

### SUMMARY OF THE INVENTION

The present invention provides a composition for anti-obesity property with health-restorative and health-promotional benefits to humans comprising the extract of Chlorophytum species, and more particularly, Chlorophytum arundinaceum. Throughout the specification, unless otherwise indicated reference to the composition means the chemical composition or chemical makeup of the plant extract being referenced.

The bio-active components of the present invention responsible for the anti-obesity property with health-restorative and health-promotional benefits comprises one or more of the following chemical constituents:
a) Spirosta-steroidal saponins comprising diosgenin, tigogenin, neotigogenin and sarasasapogenin as the major genin components and mono-, di- and oligosaccharides, comprising glucose, rhamnsoe, arabinose, galactose and xylose as glycosidic components;
b) Spirosta-steroidal glycoalkaloids comprising mainly solasodine and tomatidine as the steroidal alkaloidal aglycones, and mono-, di- and oligosaccharides, comprising glucose, rhamnose, arabinose, galactose and xylose as glycosidic components; and
c) Galacto-glucan oligosaccharides. Optionally, they contain oligosaccharides comprising glucose, rhamnose, arabinose, galactose and xylose.

The bio-active components of the present invention responsible for anti-obesity property with health-restorative and health-promotional benefits may also contain, one or more of the following additional constituents:
a) Amino acids. For example, alanine, glycine, valine, isoleucine, proline lysine, serine, threonine, aspartic acid, ornithine, glutamic acid, phenyl alanine, tyrosine, arginine;
b) Phytosterols, sitosterols and sterols. For example, chlolest-7-en-6-one, 3-(acetoxy)-9-hydroxy, cholesta-7,9(11)-dien-3-ol, 4,4-dimethyl, cholest-8(14)-en-3-one, cholest-8(14)-en-3-ol, ergosterol, androstan-17-one-3-hydroxy. (Surprisingly, the presence of androstane derivatives in all other/wild crafted species of chlorophytum was not found); and
c) Reductone and related derivatives. For one example, 2-ketogluconolactone, 2-ketogluconolactone-6-phosphate may be a constituent.

The composition of the present invention can also include other active ingredient(s), for example, one or more antioxidants, one or more adaptogens, vitamins, minerals, or one or more plant extracts other than Chlorophytum species, and mixtures thereof.

The anti-obesity composition and formulations thereof of the present invention also provides cardiovascular relief due to decrease in fasting sugar, cholesterol, triglycerides, low-density lipid, VLDL and serum cortisol with concomitant increase in hemoglobin, serum high-density lipid and dehydroepiandrosterone (DHEA) in stress subjects after treatment. The composition and formulation of the present invention provides weight-loss to stressed humans as well by reducing cortisol-induced weight gain. The present invention also provides a means of protecting target organs against stress-induced damage.

In another aspect, the present invention provides a suitable delivery system for the composition of the present invention. Such delivery systems include, but are not limited to, a nutritional beverage, nutritional bar, powder, coffee, tea, soft drink, capsule, tablet, granule, pudding, yoghurt, candy, cookie, cereal, and the like.

In another aspect, the present invention provides a pharmaceutical, veterinary or nutritional formulation. The formulation of the present invention comprises extracts of Chlorophytum species, and more specifically, the Chlorophytum arundinacceum extract composition that is present in an amount of about 0.05% to about 99% by weight is desired.

Also described are pharmaceutical formulations comprising extracts of Chlorophytum species, and more specifically, the Chlorophytum arundinacceum extract composition. The pharmaceutical formulation can be in the form of a tablet, syrup, elixir or capsule, or any other pharmaceutically acceptable form.

Also described is a nutritional formulation comprising extracts of Chlorophytum species, and more specifically, Chlorophytum arundinacceum extract composition. The nutritional formulation contains about 0.05% to about 99% of the Chlorophytum arundinacceum extract composition by weight.

Also described is a veterinary formulation comprising extracts of Chlorophytum species, and more specifically, the Chlorophytum arundinacceum extract composition. The veterinary formulation contains about 0.05% to about 99% of the Chlorophytum arundinacceum extract composition by weight.

### DETAILED DESCRIPTION OF THE PRESENT INVENTION

Reference will now be made in greater detail to a preferred embodiments of the invention.

An average person has about 40 billion fat cells in the body. When calorie intake exceeds expenditure, fat cells swell to as much as six times their minimum size, and begin to multiply from 40 billion in an average adult up to 100 billion. Fat requires a copious supply of blood in tiny capillaries (compared with an equal weight of lean muscle, which is supplied by larger blood vessels); this puts a strain on the cardiovascular system. Obesity creates wear on the joints leading to osteoarthritis. The accumulation of fat around the windpipe can interfere with breathing when muscles relax in sleep. And fat discourages exercise. Fat cells are continuously sending messages through the bloodstream to the rest of the body and their adverse signals can ruin human health. It is almost impossible to destroy them without impairing the metabolic functions of the body.

Obesity researchers are up against a phenomenally complex and robust system, devised by evolution precisely for the purpose of hoarding fat against the certainty of future famine. The search for a simple cure for obesity failed for decades. Surprisingly, it was found that an extract of Chlorophytum arundinaceum, has significant anti-obesity, health-restorative and health-promotional benefits to animals, and in particular, humans.

Of about eight species under the name of Safed Musli reported in India, Chlorophytum arundinaceum is richest in terms of variety and content of the bioactives of Safed Musli and is the preferred variety for practicing the invention. Additionally, a cultivated variety is preferred over a wild crafted variety. However, bioactives are present in all the reported varieties and all may be used in the practice of the present invention.

The major bioactive constituents of Safed Musli, and in particular of Chlorophytum arundinaceum, are disclosed. The uses of the extracts of Safed Musli, and in particular of Chlorophytum arundinaceum, for medicinal purposes are also disclosed. More specifically, the biological effects of the bioactive constituents on food intake, body weight, plasma cholesterol and triglyceride levels, immuno-modulatory effect(s), AAPH induced haemolysis of RBC, and lipid peroxidation are disclosed. Because of its impact on these biological indicators, the bioactive constituents of Safed Musli, and in particular of Chlorophytum arundinaceum are useful for treating a variety of health conditions including, but not limited to, obesity.

The present invention is now exemplified in detail by using Chlorophytum arundinaceum plant.

1. Major Bioactives of Chlorophytum arundinaceum Baker

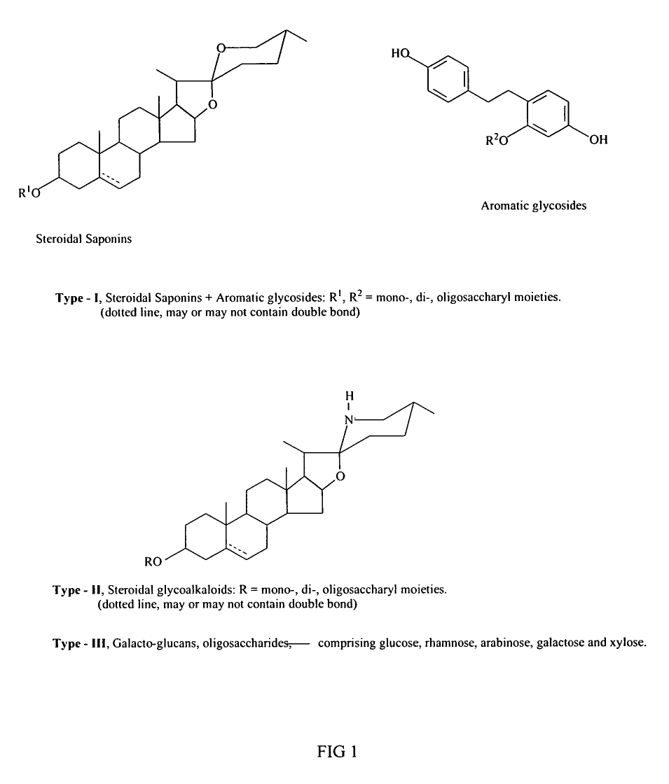

Three distinct types of compounds (typically Type-1,10-30%; Type-I, 0.5-5% and Type-III, 15-40%) (FIG. 1) can be isolated and characterized from the fresh tuber-roots of a cultivated variety of Chlorophytum arundinaceum. Extraction is preferably performed with aqueous-methanol, but can be performed with other solvents or solvent systems, and is followed by extensive column chromatography. Characterization, while not necessary to practice the present invention, can be accomplished with comprehensive HPTLC, HPLC and GC-MS analyses (using authentic markers, where possible) for the isolation, characterization and quantification of the extracted compounds. The types of bioactive constituents that are extracted by the method of the present invention are as follows:

Type-1: constitutes spirosta-steroidal saponins comprising diosgenin, tigogenin, neotigogenin and sarsasapogenin as the major genin components and mono-, di- and oligosaccharides, comprising glucose, rhamnose, arabinose, galactose and xylose as glycosidic components.

Optionally, Type-I may constitute an additional type of constituents belonging to phenolic dibenzyls. They are reduced resveratrol-type compounds having potent anti-oxidant and immuno-modulatory activities.

Type-II: constitutes spirosta-steroidal glycoalkaloids comprising mainly solasodine and tomatidine as the alkaloidal aglycones, and mono-, di- and oligosaccharides, comprising glucose, rhamnose, arabinose, galactose and xylose as glycosidic components.

Type-III: constitutes galacto-glucan oligosaccharides. Optionally, they contain oligosaccharides comprising glucose, rhamnose, arabinose, galactose and xylose.

2. Biological Effects of the Chemical Constituents of Chlorophytum arundinaceum

The results of administration of the Type-I saponins or the total extractives (comprising Type-I to -III compounds) of Chlorophytum arundinaceum on the attendant changes in the food intake and body weight of albino rats (270±20 grams) are given in Table-1. Administration of haloperidol causes obesity as it appreciably increases food intake and induces rapid weight gain. The feeder contained at least three times more pellets than are normally consumed. Food intake and the body weight were assessed for one week in all rats prior to initiation of administration of the test compound(s). Both Type-I saponins and the total extractives annulled the gluttonous craving for food due to haloperidol with appreciable decrease in weight gain, compared to the haloperidol group. The Type-I saponins administered alone, however, exhibited a better effect than the total extractives (Table-1). Sibutramine is used as a recognized anorexic drug.

TABLE 1
Effect of Chlorophytum arundinaceum constituents on food intake and
body weight in albino rats.

| | % change in | % change in |
|---|---|---|
| Group/treatment | food intake^{a} | body weight^{b} |
| control^{c} | +24.08 | +22.10 |
| Haloperidol^{d} | +45.32 | +35.13 |
| Sibutramine^{e} + Haloperidol^{d} | −0.37 | +0.28 |
| Type-I saponins^{f} + Haloperidol^{d} | +26.11 | +18.34 |
| Total aqueous-methanol | +30.77 | +20.16 |
| Extractives^{f} + Haloperidol^{d} | | |

n = 10-12
^{a}during the 21-day study period, compared to the initial value
^{b}On day-21, compared to the initial value
^{c}1 ml of water, p.o., once daily for 21 days
^{d}Haloperidol (Seranace), 20 mg/Kg, i.p., once daily for 21 days
^{e}Sibutramine (Cipla), 10 mg/Kg, p.o., once daily for 21 days
^{f}100 mg/Kg, p.o. (in water), once daily for 21 days

The findings of administration of Type-I saponins, Type-II steroidal glycoalkaloids, and the total extractives (aqueous-methanol) of Chlorophytum arundinaceum on plasma cholesterol and triglyceride levels of obese albino rats (270±20 grams) are given in Table-2. The results indicate that the Type I and Type III compounds, alone or in combination, have a beneficial effect on the total cholesterol triglycerides levels in plasma. The results also indicate that the Type-III compounds (oligosaccharides) provide additive effect to the Type-I compounds in respect of decrease in the plasma cholesterol and triglyceride levels.

TABLE 2
Effects of Chlorophytum arundinaceum constituents on plasma
biochemical parameters (mg/dl) of albino rats

| | Total | |
|---|---|---|
| Group/treatment^{a} | cholesterol | Triglycerides |
| control^{b} | 148.1 ± 7.4 | 48.7 ± 5.0 |
| Type-I saponins | 98.2* ± 3.7 | 32.1** ± 2.4 |
| Type-II alkaloids | 105.6* ± 7.9 | 38.5* ± 6.2 |
| Aqueous-methanolic | 90.8** ± 4.7 | 30.9** ± 3.7 |
| extract^{a,c} | | |

^{a}The test compounds were administered 100 mg/Kg, p.o. (in water, 1 ml), once daily for 14 days
^{b}1 ml water, p.o., once daily for 14 days
^{c}comprising saponins (17.2%), steroidal alkaloids (2.1%) and oligosaccharides (22.5%) values are expressed as mean ± SEM of 6-8 rats in each group; * and ** indicate p < 0.05 and 0.01, respectively, compared to control group (ANOVA followed by Newman-Keuls test).

In another aspect, the immuno-modulatory effect(s) of the constituents of Chlorophytum arundinaceum was assessed, among other determinations, by their capacity to attenuate the cold-immobilization stress-induced elevated level of plasma corticosterone. The results are given in Table-3.

TABLE 3
Effect of Chlorophytum arundinaceum constituents on stress-induced
elevated level of corticosterone.

| | Nature of chemical | Plasma |
|---|---|---|
| Treatment | Compounds/dose^{a} | corticosterone (μg/dl) |
| Unstressed control | — | 28.77 ± 4.10 |
| Stressed control | — | 111.34 ± 7.32 |
| Type-III | galacto-glucans and | 68.92 ± 8.82 |
| (oligosaccharides) | other oligosaccharides | |
| Aqueous-methanol | saponins, steroidal | 71.33 ± 7.70 |
| extractives | glycoalkaloids, | |
| | oligosaccharides^{b} | |

^{a}100 mg/Kg, (in 1 ml water), p.o. once daily for 7 days
^{b}for % composition, see Table-2 (footnote c)

In another aspect, the effects of the chemical constituents of Chlorophytum arundinaceum were evaluated as protector against lipid peroxidation and also their per se anti-haemolytic effect. Unlike other bioactive saponins, which suffer from the adverse effect of per se haemolytic activity (even the reputed adaptogen Panax ginseng suffers from its haemolytic activity), Chlorophytum arundinaceum saponins (Type-I compounds) particularly in higher doses, produced anti-haemolytic activity (FIG. 2).

3. Anti-Haemolytic Effect and Inhibition of Lipid Peroxidation by Chlorophytum arundinaceum Root Extractives and the Contained Saponins (Type-I)

a) Inhibition of AAPH-Induced Haemolysis:

Membrane protecting activity was observed for Chlorophytum arundinaceum constituents against AAPH [2,2′-Azobis (2-methylpropionamidine) dihydrochloride]-induced haemolysis. AAPH generates oxygen free radicals (OH) which, in turn, disrupt RBC membrane resulting in haemolysis. Agents that inhibit AAPH-induced haemolysis possess strong oxygen radical scavenging activity. Different doses (20-600 μg/ml) of the Chlorophytum arundinaceum constituents were incubated at 37° C. for 90 minutes with 10% RBC suspension (from mice, goat and human blood) in presence of AAPH (200 mM). The IC₅₀ values to protect against haemolysis (AAPH) were calculated. The results are depicted in Table 4.

TABLE 4
Comparative IC₅₀ values of Chlorophytum arundinaceum root extract and
saponin type I in AAPH induced haemolysis of RBC of different species

| | IC₅₀ Value | | |
|---|---|---|---|
| | Mice RBC | Goat RBC | Human RBC |
| Chlorphytum arundinaceum | 110 | 81.7 | 86.5 |
| root extract | | | |
| Chlorphytum saponins Type 1 | 43 | 38.8 | 35.5 |

b) Inhibition of Spontaneous Lipid Peroxidation:

Chlorophytum arundinaceum constituents (Type-I saponins and the aqueous-methanol extractives), in the dose range of 50-500 μg/ml, were tested. Mice brain homogenate (25%) was allowed to spontaneous lipid peroxidation in the presence and absence of different doses of the two test compounds. Each mixture was put to a shaking water bath for 60 minutes. The amount of malondialdehyde (MDA) produced was estimated by developing color using 1% thiobarbituric acid (TBA) and incubated for one hour at 85-90° C. The developed pink color was measured at 532 nm using the formula:

$$\frac{OD of control - OD of sample}{OD of control} \times 100$$

The respective IC₅₀ values are depicted in Table 5.

TABLE 5
Comparative IC₅₀ values of Chlorophytum arundinaceum root extract and
saponin type I in spontaneous lipid peroxidation test

| | |
|---|---|
| Chlorphytum arundinaceum | 448 |
| root extract | |
| Chlorphytum saponins Type 1 | 402 |

4. Mechanism of Biological Action of Chlorophytum arundinaceum (Chemical Spectroscopic and Ex-Vivo Evidence)

While not bound by theory an investigation into the mechanism of biological action of Safed Musli was performed. The total aqueous methanol extractives and the Type-I saponins of Chlorophytum arundinaceum produced sparingly water-soluble stable adducts with both cholesterol and triglycerides. For the adduct formation, optimum ratios of the two adduct-components, e.g. Chlorophytum arundinaceum constituents: cholesterol or triglycerides were found to be in the range of 4:1 to 2:1. The adduct formation and their tenacious attachment to each other were established by HPTLC [CAMAG TLC evaluation software; precoated plates Sigel Merck 60F254; solvent, ethyl acetate—formic acid—acetic acid—water, 100:11:11:27; visualizing and scanning after Libermann-Burchard reagent spray, and heating] and by GC-MS (as OTMS derivatives) analyses, using respective markers. These observations suggest that excess of systemic cholesterol/triglycerides, in tissues or from food materials, can be excreted out as adducts on treatment with Chlorophytum arundinaceum (Type-I saponins or total extractives). Indeed, administration of the Type-I saponins (20-100 mg/Kg, p.o.) to albino rats, fed with cholesterol and triglyceride-rich diets, caused significant dose-dependant fecal elimination (10-40% increase over the control values) of cholesterol and triglycerides as adducts. The fecal matter was extracted with n-butyl alcohol and the butanol extractives before and after acidic hydrolysis was subjected to HPTLC and GC-MS analyses. Since glycosidation greatly reduces the toxicity of steroidal alkaloids and increases their systemic efficacy, it is expected that the Type-II glycoalkaloids of Chlorophytum arundinaceum will be ideal candidates for the above biological effects.

5. Anti-Obesity. Health-Restorative and Health-Promotional Compositions from Other Plant Sources

In another aspect of the invention, additional plants containing the same or similar bioactives as found in Chlorophytum arundinaceum are as follows:
1. Chlorophytum borivilianum
2. Tribulus terrestris L. (Zygophyllaceae)
3. Solanum hispidum Pers. (Solanaceae)
4. Trigonella foenum-graecum L. (Papilionaceae)
5. Digitalis purpurea L. (Scrophulariaceae)
6. Dioscorea floribunda Mart & Gal. (Dioscoreaceae)
7. Costus speciosus Sm. (Zingiberaceae)
8. Asparagus racemosus Willd. (Liliaceae)

Extracts of these plants may be used in the same manner as Chlorophytum arundinaceum to produce the health benefits of the present invention.

The anti-obesity effect (Table 1) and the unique ability of the bioactive agents (FIG. 1) of Chlorophytum arundinaceum and other species to provide protection against AAPH-induced haemolysis (FIG. 2) (unlike other saponins which are per se haemolytic) and against lipid peroxidation (FIG. 3) make them useful to control obesity as well as for health-restorative and health-promotional purposes.

6. Other Actives

The inventive composition may optionally include one or more of the following active ingredients:
a) Antioxidants—One or more antioxidants, for example, alpha lipoic acid, coenzyme Q, tocopherols, carnosine, carnithin, acetyl carnithin, natural polyphenolics obtained from Phyllanthus species, Terminalia species, Grenn tea, Grape antioxidant, pine antioxidants, reductones, e.g., 2-ketogluconolactones and related compounds.
b) Adaptogens—One or more adaptogens, for example, Withania somnifera, purified Shilajit, Panax Ginseng, Siberian ginseng, Physalis peruviana and phytosteroids occurring there and, produced thereof.
c) Amino acids—Present in C. arundinaceum or added to the present inventive composition. Examples are one or more amino acids comprising of lysine, proline, ornithine, glutamine, phenylalanine, tyrosine, arginine, etc to augment energy synthesis.
   7. Pharmaceutical Formulations

The extracts of the present invention can be incorporated into an acceptable pharmaceutical or medicinal formulation with nutritionally or veterinary acceptable excipients. The formulation can be administered to a mammal, particularly a primate, and more particularly, a human in an effective dose to reduce obesity, which may also reduce cholesterol, triglycerides and low-density lipids. In the preferred embodiment, the formulation is administered once or twice a day.

In further embodiments, an adaptogen may be included in the formulation. Other additional components may also be added, including but not limited to the following: antioxidants; effective amounts of transition and/or trace metals; additional anti-obesity ingredients; and phenolic dibenzyl phenolic dibenzyl, for example, reduced resveratrol-type compounds having potent anti-oxidant and immuno-modulatory activities.

The transition or trace metals preferably include one or more of copper, chromium, zinc, selenium and/or metal ions ranging from 1 to about 500 ppm levels. Additional anti-obesity ingredients preferably include one or more of conjugated linoleic acid, or bitter orange, or hydroxycitric acid, or chitosan, or startch blockers or dehydroepiandesterone (DHEA) or adaptogens.

It is to be understood that the above-described embodiments are illustrative of only a few of the many possible specific embodiments, which can represent applications of the principles of the invention. Numerous and varied other arrangements can be readily devised in accordance with these principles by those skilled in the art without departing from the spirit and scope of the invention.

### EXAMPLES OF FORMULATIONS

The following describes examples of formulations of the present invention that incorporate the inventive extract composition.

Beverage Mixes

### Example 1

Sugar-free red punch powdered soft drink mix with anti-obesity, health-restorative and health promotional benefits: serving size: 1.4 gm mixed with 8 oz of water.

| Ingredient | % |
|---|---|
| Citric Acid | 38.76 |
| Chlorophytum species or Chlorophytum arundinaceum extracts | 8.92 |
| Maltodextrin, M100 | 18.82 |
| Flavors % Colors | 17.98 |
| NutraSweet ® brand Sweetener | 8.05 |
| Tricalcium phosphare | 5.15 |
| Potassium citrate | 1.84 |
| Vitamin C | 0.48 |
| TOTAL | 100.0 |

Process: Mix all the powdered ingredients in a suitable blender for 15 minutes.

### Example 2

Sugar-free iced tea powdered soft drink mix with anti-obesity, health-restorative and health promotional benefits: serving size: 1.3 g mixed with 8 oz of water.

| Ingredient | % |
|---|---|
| Tea Powder | 50.68 |
| Chlorophytum species or Chlorophytum arundinaceum extracts | 5.00 |
| Citric Acid | 27.83 |
| Maltodextrin, M100 | 9.28 |
| NutraSweet ® brand Sweetener | 6.03 |
| Flavors % Colors | 1.18 |
| TOTAL | 100.0 |

Process: Mix all the powdered ingredients in a suitable blender for 15 minutes.

Ready to Drink Beverages

### Example 3

30% Orange juice beverage with anti-obesity, health-restorative and health promotional benefits: serving size: 8 oz.

| Ingredient | % |
|---|---|
| Treated Water | 93.368 |
| Citric Acid | 0.170 |
| NutraSweet ® brand Sweetener | 0.040 |
| Chlorophytum arundinaceum and Withania somnifera Extracts | 0.002 |
| (1:1) | |
| Potassiun citrate | 0.020 |
| Orange juice concentrate | 5.620 |
| Orange flavor | 0.090 |
| Peach Flavor | 0.490 |
| Color (1% solution) | 0.200 |
| TOTAL | 100.00 |

Process:
1. Blend water with Citric acid and Chlorophytum arundinaceum and Withania somnifera Extracts (1:1) under agitation
2. Add the sweetener and mix well until the sweetener dissolves.
3. Add other ingredients and mix thoroughly
4. Pasteurize as normal plant practice.
5. Cool and pack.

### Example 4

Light lemon iced tea with anti-obesity, health-restorative and health promotional benefits.

| Ingredient | % |
|---|---|
| Treated water | 99.395 |
| Potassium citrate | 0.010 |
| Chlorophytum or C. arundinaceum extracts | 0.025 |
| NutraSweet ® brand Sweetener | 0.050 |
| Lemon Flavor | 0.120 |
| Tea base | 0.230 |
| Caramel color | 0.170 |
| TOTAL | 100.00 |

Process:
1. Blend water with Potassium citrate and Chlorophytum or C. arundinaceum extracts under agitation
2. Add the sweetener and mix well until the sweetener dissolves.
3. Add other ingredients and mix thoroughly
4. Pasteurize as normal plant practice.
5. Cool and pack.
   Carbonated Soft Drinks

### Example 5

Low calorie carbonated soft drink with anti-obesity, health-restorative and health promotional benefits: serving size: 8 oz.

| Ingredient | % |
|---|---|
| Treated alkaline-free water | 42.360 |
| Chlorophytum species or Chlorophytum arundinaceum extracts | 0.010 |
| Sodium benzoate | 0.100 |
| High Fructose Corn Sweetener, HFCS 55 | 55.790 |
| Phosphoric acid, caffeine solution | 0.270 |
| Artificial sweetener | 0.030 |
| Cola Flavor | 1.440 |
| TOTAL | 100.00 |

Process:
1. Add treated water to syrup tank, reserving sufficient water for rinsing containers.
2. Completely dissolve sodium benzoate and Chlorophytum species or Chlorophytum arundinaceum extracts prior to addition of other ingredients
3. Add HFCS 55 and mix well.
4. Add acid, acid solution or acid/caffeine blend. Rinse container. Allow to disperse completely.
5. Slowly add the artificial sweetener with gentle mixing and mix for 30 minutes.
6. Add Cola flavor and mix well.

### Example 6

Regular carbonated soft drink with anti-obesity, health-restorative and health promotional benefits: serving size: 8 oz.

| Ingredient | % |
|---|---|
| Treated alkaline-free water | 42.390 |
| Chlorophytum species or Chlorophytum arundinaceum extracts | 0.010 |
| Sodium benzoate | 0.100 |
| Sucrose | 55.790 |
| Phosphoric acid, caffeine solution | 0.270 |
| Cola Flavor | 1.440 |
| TOTAL | 100.00 |

Process:
1. Add treated water to syrup tank, reserving sufficient water for rinsing containers.
2. Completely dissolve sodium benzoate and Chlorophytum species or Chlorophytum arundinaceum extracts prior to addition of other ingredients.
3. Add sucrose and mix well.
4. Add acid, acid solution or acid/caffeine blend. Rinse container. Allow to disperse completely.
5. Add Cola flavor and mix well.
   Meal Replacement Beverage Mix

### Example 7

Chocolate-flavored meal replacement beverage mix with anti-obesity, health-restorative and health promotional benefits.

| Ingredient | % |
|---|---|
| Sucrose | 39.00 |
| Whey protein concentrate, 34% | 18.80 |
| Dutch processed Cocoa, 16-18% fat | 11.50 |
| Corn syrup solids | 11.50 |
| Sodium caseinate | 11.00 |
| Chlorophytum species or Chlorophytum arundinaceum extracts | 0.20 |
| Calcium caseinate | 5.00 |
| Vitamin/Mineral Premix (Adjusted to provide 25-30% of daily | 1.00 |
| recommended intake based on 2000 kcal diet) | |
| Vanilla extract | 0.90 |
| Lecithin | 0.80 |
| Xanthan gum | 0.20 |
| Carboxy Methyl Cellulose | 0.10 |
| TOTAL | 100.0 |

Process:
1. Dry blend all the ingredients and package as desired.
2. To serve, mix 40 g of the dry mixture in 225 ml of milk.
   Sports Beverage

### Example 8

Sports beverage with anti-obesity, health-restorative and health promotional benefits: serving size: 220 g

| Ingredient | % |
|---|---|
| Purified Water | 76.33 |
| Maltodextrin, 18DE | 10.00 |
| Fructose | 9.15 |
| 80% Whey protein concentrate (WPC80) | 3.60 |
| Chlorophytum species and purified Shilajit (5:1) | 0.20 |
| Citric Acid | 0.56 |
| Flavor | 0.09 |
| Sodium citrate dihydrate | 0.06 |
| Color | 0.01 |
| TOTAL | 100.0 |

Process:
1. Add water to a large mixing tank at 15-25° C.
2. With good agitation, add WPC80, avoiding entrapment of air. Allow mixture to sit for 15-30 minutes so that WPC80 can become hydrated
3. Mix in fructose, maltodextrin, sodium citrate and Chlorophytum species and purified Shilajit (5:1) with good agitation.
4. Add flavor and color. Allow to hydrate for 10 minutes
5. Adjust pH to 3.5-3.7 using a 50% solution of an appropriate acid while continuously mixing.
6. Hot-fill containers.
7. Cool beverages immediately.
   Coffee and Tea

### Example 9

Cappuccino mix with anti-obesity, health-restorative and health promotional benefits: serving size: 26 g

| Ingredient | % |
|---|---|
| Sugar | 41.45 |
| Nonfat dry milk | 25.40 |
| Creamer | 22.80 |
| Chlorophytum species or Chlorophytum arundinaceum extracts | 0.160 |
| Instant Coffee | 6.15 |
| Cocoa | 2.65 |
| Xanthan Gum | 0.70 |
| Natural Flavor | 0.45 |
| Salt | 0.20 |
| TOTAL | 100.0 |

Process:
1. Mix sugar, salt, cocoa and Chlorophytum species or Chlorophytum arundinaceum extracts until well blended.
2. Add Instant Coffee and xanthan gum. Mix.
3. Add remaining ingredients and mix until well blended.

### Example 10

Tea with anti-obesity, health-restorative and health promotional benefits: serving size: 31 g

| Ingredient | % |
|---|---|
| Sugar | 42.00 |
| Whole Dry Milk | 16.40 |
| Honey Powder | 13.50 |
| Nonfat Dry Milk | 11.25 |
| Creamer | 9.60 |
| Chlorophytum species or Chlorophytum arundinaceum extracts | 0.50 |
| Whey Mineral Concentrate/Milk Calcium | 2.70 |
| Black Tea | 1.90 |
| Natural and Artificial Flavor | 1.20 |
| Spice Blend 9Cardamom, Clove, Anise, Cinnamon, Ginger) | 0.60 |
| Lactoferrin | 0.35 |
| TOTAL | 100.0 |

Process:
1. Blend sugar, honey powder, spice blend, Chlorophytum species or Chlorophytum arundinaceum extracts and tea until well mixed.
2. Add diary ingredients and mix until well dispersed.
3. Add remaining ingredients and mix well.
4. Package to a net weight of 31 g.

### Example 11

Instant coffee with anti-obesity, health-restorative and health promotional benefits: serving size: 5 gm mixed in 200 ml hot water.

| Ingredient | % |
|---|---|
| Instant Coffee | 99.20 |
| Chlorophytum species or Chlorophytum arundinaceum and | 0.80 |
| Withania somnifera extracts (1:1) | |
| TOTAL | 100.0 |

Process:
1. Mix Chlorophytum species or Chlorophytum arundinaceum and Withania somnifera extracts (1:1) with Coffee extract until thoroughly mixed.
2. Instantise by freeze-drying or another appropriate method.
3. Pack into bottles or aluminum pouches.

### Example 12

Percolated coffee with anti-obesity, health-restorative and health promotional benefits: serving size: 200 ml.

Ingredient

| | | |
|---|---|---|
| Water | 3.9 | L |
| Ground Coffee | 96.80 | g |
| Chlorophytum species or Chlorophytum arundinaceum and | 3.20 | g |
| Withania somnifera extracts (1:1) | | |
| TOTAL | 100.0 | g |

Process:
1. Mix Chlorophytum species or Chlorophytum arundinaceum and Withania somnifera extracts (1:1) with Ground Coffee until thoroughly mixed.
2. Add the above mixture to a coffee filter in a coffee maker and add 3.9 L of water and brew.
3. Mix the percolated coffee well before serving.
4. Alternatively, Chlorophytum species or Chlorophytum arundinaceum and Withania somnifera extracts (1:1) and the Ground Coffee may be added separately to the coffee filter and then brewed with water.
   Bakery Formulations

### Example 13

Chocolate chip cookies with anti-obesity, health-restorative and health promotional benefits: serving size: 25 g.

| Ingredient | % |
|---|---|
| Chocolate Chips | 26.50 |
| All Purpose Flour | 24.16 |
| Chlorophytum species or Chlorophytum arundinaceum and | 0.50 |
| purified Shilajit (1:1) | |
| Butter | 17.47 |
| Sugar | 11.05 |
| Brown Sugar | 10.06 |
| Eggs | 7.86 |
| Nonfat Dry Milk | 2.14 |
| Salt | 0.53 |
| Baking Soda | 0.43 |
| Vanilla Extract | 0.30 |
| TOTAL | 100.0 |

Process:
1. Cream butter with sugar.
2. Add vanilla and eggs and mix.
3. Add dry ingredients and mix until blended.
4. Add chocolate chips.
5. Bake at 190° C. for 8-10 minutes.

### Example 14

White bread with anti-obesity, health-restorative and health promotional benefits: serving size: 35 g.

| Ingredient | % |
|---|---|
| Bread Flour | 54.14 |
| Chlorophytum arundinaceum, Taste | 0.06 |
| Masked, 33% | |
| Water | 37.20 |
| Sugar | 3.30 |
| Shortening | 2.00 |
| 80% Whey Protein Concentrate | 1.10 |
| Salt | 1.00 |
| Yeast | 0.70 |
| Whole Dry Milk | 0.50 |
| TOTAL | 100.0 |

Process:
1. Combine and mix all the dry ingredients on low speed for 3 minutes
2. Add shortening and water, mixing on low speed for 2 minutes.
3. Mix on medium speed for 11-22 minutes or until dough passes gluten test (when pulled, dough stretches with no rough tearing.)
4. Proof dough until double in size, about 1 hour.
5. Shape into loaves and place in greased loaf pans. Allow to full proof until double, about 30-45 minutes.
6. Bake at 204° C. for 20-30 minutes.

### Example 15

Coffee cake with anti-obesity, health-restorative and health promotional benefits: serving size: 90 g.

| Ingredient | % |
|---|---|
| All Purpose Fluor | 31.40 |
| Brown Sugar | 23.00 |
| Water | 20.00 |
| Butter | 11.00 |
| Eggs | 7.50 |
| Chopped Nuts | 4.00 |
| Nonfat Dry Milk | 2.00 |
| Chlorophytum species, Taste-masked, 66% | 0.100 |
| Baking Powder | 0.55 |
| Salt | 0.20 |
| Baking Soda | 0.20 |
| Cinnamon | 0.05 |
| TOTAL | 100.0 |

Process:
1. Combine flour, brown sugar, salt and Chlorophytum species, Taste-masked, 66%. Cut into shortening and mix until crumbly; set aside ¼ cup crumb mixture.
2. Add baking powder and baking soda to remaining crumb mixture. Add cinnamon, butter, milk and egg and mix well.
3. Spread batter onto a greased 8×8×2 inch baking pan.
4. Stir together reserved crumbs and nuts; sprinkle on batter.
5. Bake at 176° C. for 30-35 minutes.

### Example 16

Energy bar with anti-obesity, health-restorative and health promotional benefits: serving size: 50 g.

| Ingredient | % |
|---|---|
| Brown Rice Syrup | 21.10 |
| Brown Rice Crisp Cereal | 14.10 |
| Old Fashioned Rolled Oats | 10.60 |
| Quick Rolled Oats | 10.60 |
| Water | 10.60 |
| Dried Cherries | 8.80 |
| Cherry-Flavored Dried Cranberries | 7.10 |
| Plum Paste | 6.50 |
| Whey Protein Isolate | 4.80 |
| Chlrophytum species or Chlorophytum arundinaceum and | 1.00 |
| Withania somnifera extracts (1:1) | |
| Unsalted Butter | 3.40 |
| Glycerine | 0.80 |
| Black Cherry Flavor | 0.50 |
| Sodium Bicarbonate | 0.10 |
| TOTAL | 100.0 |

Process:
1. Combine the first ten ingredients, except water, in the bowl of a large mixer. Mix on low speed for 2 minutes.
2. Add butter, black cherry flavor and glycerine and mix on low speed for 1 minute.
3. Add water and mix on low speed for ½ minutes.
4. Sheet bars to 11 mm thickness and cut into 1½″×1½″ pieces.
5. Place on parchment lined pans so that they are not touching each other.
6. Bake at 204° C. for 7 minutes.
   Prepared Foods

### Example 17

Salad dressing, dry mix, with anti-obesity, health-restorative and health promotional benefits: serving size: 2.5 g.

| Ingredient | % |
|---|---|
| Salt | 13.49 |
| Fructose | 12.48 |
| Powdered Vinegar | 12.50 |
| Dry Sweet Whey | 12.26 |
| Sugar | 11.15 |
| Fat Replacer Instant Starch | 12.15 |
| Starch | 4.42 |
| Garlic Powder | 3.85 |
| Chlorophytum species or Chlorophytum | 5.00 |
| arundinaceum and Withania somnifera | |
| extracts (1:1) | |
| Citric Acid | 3.31 |
| Dry Mustard | 1.55 |
| Basil | 1.55 |
| Parsley | 1.24 |
| Xanthan Gum | 1.10 |
| Onion Powder | 0.93 |
| Black Pepper | 0.93 |
| Guar Gum | 0.77 |
| Paprika | 0.62 |
| Titanium Dioxide | 0.33 |
| Oregano | 0.31 |
| Dill | 0.06 |
| TOTAL | 100.0 |

Process:
1. Mix together all the ingredients thoroughly.
2. Package 50 g into individual packages.
3. Mix 50 g dry mix with ½ cup water using whisk or an electric mixer, or shake in a bottle.
4. Add ½ cup skim milk and mix vigorously.
5. Store in refrigerator for at least 1 hour before serving.

### Example 18

Cream of mushroom soup with anti-obesity, health-restorative and health promotional benefits: serving size: 240 g.

| Ingredient | % |
|---|---|
| Stage I | |
| Water | 13.95 |
| Cream (30% Fat) | 1.85 |
| Vegetable Oil | 1.75 |
| Nonfat Dry Milk | 1.40 |
| 34% Why Protein Concentrate | 0.60 |
| Disodium Phosphate | 0.50 |
| Chlorophytum species or Chlorophytum | 0.05 |
| arundinaceum extracts | |
| Stage II | |
| Water | 19.00 |
| Diced Mushrooms | 14.00 |
| Salt | 1.80 |
| Flavor Enhancers | 1.05 |
| Flavors | 0.40 |
| Stage III | |
| Steam Condensate & Final Dilution of Water | 22.75 |
| Water to Slurry | 15.00 |
| Modified Cook-up Starch | 3.30 |
| Corn Starch | 1.60 |
| Wheat Flour | 1.00 |
| TOTAL | 100.0 |

Procedure:
Stage I: Emulsion Preparation
1. Hydrate non-fat dry milk, Chlorophytum species or Chlorophytum arundinaceum extracts and 34% whey protein concentrate in 38° C. water.
2. Add oil and cream to hydrated milk proteins and blend.
3. Homogenize to 60° C. and appropriate conditions for pressure.
   Stage II: Mushroom Preparation
1. Blanch mushrooms in formula water at 93° C. for 3-4 minutes
2. Add salt, flavors and flavor enhancers.
3. Heat with live steam to 40° C.
   Stage III: Thickener Slurry Preparation
1. Mix wheat flour, corn starch and modified starch with chilled Stage III formula water to make a slurry.
2. Add the starch slurry to the kettle of other ingredients and heat at 88° C. to gelatinize the starch.
3. Adjust to final weight with hot water, mixing thoroughly.
4. Fill cans with hot product.
   Meat Products

### Example 19

Beef patty with anti-obesity, health-restorative and health promotional benefits: serving size: 113 g.

| Ingredient | % |
|---|---|
| Beef (90% Lean) | 85.90 |
| Water | 11.50 |
| 80% Whey Protein Concentrate | 2.00 |
| Chlorophytum species or Chlorophytum | 0.10 |
| arundinaceum and purified Shilajit (2:1) | |
| Salt | 0.50 |
| TOTAL | 100.0 |

Procedure:
1. Grind meat through 9.5 to 12.7 mm plate.
2. With mixer, blend meat, 80% whey protein concentrate, Chlorophytum species or Chlorophytum arundinaceum and purified Shilajit (2:1), salt and water for 2 minutes.
3. Hold at −1 to 1° C. to prevent the fat from smearing, and to aid in patty formation.
4. Grind through 0.32 cm plate and form into patties.
   Dairy Products

### Example 20

Sour cream with anti-obesity, health-restorative and health promotional benefits: serving size: 30 g.

| Ingredient | % |
|---|---|
| Skim Milk | 64.22 |
| Whole Milk | 30.00 |
| Whey Protein Concentrate | 3.44 |
| Chlorophytum species or Chlorophytum | 0.03 |
| arundinaceum and Withania somnifera | |
| extracts (1:1) | |
| Waxy Maize Modified Cook-up Starch | 0.76 |
| Dent Modified Instant Starch | 0.75 |
| Sodium Phosphate | 0.27 |
| Titanium Dioxide | 0.27 |
| Culture | 0.20 |
| Sodium Citrate | 0.06 |
| TOTAL | 100.0 |

Procedure:
1. Mix all dry ingredients together in a bowl.
2. Place skim and whole milk together in a pan and disperse dry ingredients in milk using a mixer.
3. Heat to 85° C. and hold for 30 minutes to pasteurize.
4. Homogenize at 70° C.
5. Cool to 21° C. and inoculate with culture.
6. Incubate at 24° C. for approximately 18 hours.
7. Cool to 4° C. and store for at least 48 hours to allow starch to set and full viscosity to be developed.
   Candies

### Example 21

Gelled candies with anti-obesity, health-restorative and health promotional benefits: serving size: 40 g.

| Ingredient | % |
|---|---|
| Maltitol Syrup, DP 55 | 68.35 |
| Water, cold | 18.77 |
| Gelatin, 225 bloom, Type B | 8.50 |
| Citric Acid Solution, 50% | 1.21 |
| Chlorophytum species or Chlorophytum | 0.05 |
| arundinaceum and Withania somnifera | |
| extracts (1:1) | |
| Sorbitol Powder | 3.00 |
| Artificial Sweetener | 0.08 |
| Color | 0.02 |
| Flavor | 0.02 |
| TOTAL | 100.0 |

Procedure:
1. Disperse the gelatin in cold water and beat to 60° C. Hold until gelatin is fully hydrated and the solution deaerates.
2. Heat the maltitol syrup to 117° C., or 88% solids and cool to 100° C.
3. Mix the maltitol syrup and gelatin/water mixture from Step 1 and hold at 71° C.
4. Premix the artificial sweetener and Chlorophytum species or Chlorophytum arundinaceum and Withania somnifera extracts (1:1) with citric acid solution and add to mixture in Step 3.
5. Add color and flavor and mix.
6. Deposit in 32° C.-35° C. dry molding starch. Let stand for 2 hours, then store at 41° C. for up to 40 hours.
7. Demold and dust off the starch. Polish with a coat of 0.2-0.4% of a blend containing 98% vegetable oil and 2% beeswax, which has been melted together thoroughly.
   Pharmaceuticals

### Example 22

Cough lozenges with anti-obesity, health-restorative and health promotional benefits: serving size: 3.7 g.

| Ingredient | % |
|---|---|
| Hydrogenated Starch Hydrolysate | 98.64 |
| Corn Oil | 0.60 |
| Chlorophytum arundinaceum and other | 0.25 |
| Chlorophytum species extracts (1:1) | |
| Artificial Sweetener | 0.20 |
| Menthol | 0.17 |
| Eucalyptus Oil | 0.14 |
| TOTAL | 100.0 |

Procedure:
1. Precook liquid hydrogenated starch hydrolysate to about 118° C.
2. Pump the precooked hydrogenated starch hydrolysate through a cooking unit and cook to about 146° C.
3. Drain the cooked syrup into a vacuum chamber to decrease moisture content to about 1.5%.
4. Mix the cooked syrup with the artificial sweetener dispersed in corn oil and remaining ingredients in an in-line mixer.
5. Temper product on a tempering band, form into a rope and die cut into desired form.
6. Cool to room temperature.

### Example 23

Chewable antacid tablets with anti-obesity, health-restorative and health promotional benefits: tablet weight: 1.5 g.

| Ingredient | % |
|---|---|
| Mannitol | 47.98 |
| Calcium Carbonate | 34.00 |
| Extracts Chlorophytum species | 16.66 |
| Artificial Sweetener | 0.24 |
| Creamy Mint Flavor | 0.24 |
| Menthol | 0.08 |
| Magnesium Stearate | 0.80 |
| TOTAL | 100.0 |

Procedure:
1. Blend all the ingredients, except magnesium stearate, in a blender for 15 minutes.

2. Add magnesium stearate and blend for 5 minutes.

3. Compress into tablets with a target weight of 1500 mg and hardness of 4-6 kp.

4. Package in airtight containers.

### Example 24

Capsules with anti-obesity, health-restorative and health promotional benefits: dose: one capsule twice daily.

| Ingredient | Per Capsule, g |
|---|---|
| Chlorophytum species or Chlorophytum | 0.250 |
| arundinaceum and Withania somnifera | |
| extracts (1:1) | |
| Microcrystalline Cellulose | 0.150 |
| Syloid (Fumed Silicon Dioxide) | 0.005 |
| Croscarmellose Sodium | 0.010 |
| Stearic Acid | 0.010 |
| Size 0 Empty Gelatin Capsule | 0.100 |
| TOTAL | 0.525 |

Procedure:
1. Blend Chlorophytum species or Chlorophytum arundinaceum and Withania somnifera extracts (1:1), Microcrystalline Cellulose, Croscarmellose Sodium and Syloid (screened through 30 mesh) in a suitable blender for 15 minutes.
2. Screen Stearic Acid through a 30 mesh, add to the above blender and mix for 5 minutes.
3. Fill into capsules with a target fill weight of 0.425 g.
4. Polish the capsules.

### Example 25

Extra-strength capsules with anti-obesity, health-restorative and health promotional benefits: dose: one capsule twice daily.

| Ingredient | Per Capsule, g |
|---|---|
| Chlorophytum species or Chlorophytum | 0.500 |
| arundinaceum extracts | |
| Microcrystalline Cellulose | 0.150 |
| Syloid (Fumed Silicon Dioxide) | 0.005 |
| Croscarmellose Sodium | 0.010 |
| Stearic Acid | 0.010 |
| Size 00 Empty Gelatin Capsule | 0.120 |
| TOTAL | 0.795 |

Procedure:
1. Blend Chlorophytum species or Chlorophytum arundinaceum extracts (1:1), Microcrystalline Cellulose, Croscarmellose Sodium and Syloid (screened through 30 mesh) in a suitable blender for 15 minutes.
2. Screen Stearic Acid through a 30 mesh, add to the above blender and mix for 5 minutes.
3. Fill into capsules with a target fill weight of 0.675 g.
4. Polish the capsules.

### Example 26

Tablets with anti-obesity, health-restorative and health promotional benefits dose: one tablet twice daily.

| Ingredient | Per Tablet, g |
|---|---|
| Chlorophytum species or Chlorophytum | 0.300 |
| arundinaceum and Withania somnifera | |
| extracts (1:1) | |
| Purified Shilajit | 0.025 |
| Microcrystalline Cellulose | 0.150 |
| Syloid (Fumed Silicon Dioxide) | 0.005 |
| Croscarmellose Sodium | 0.010 |
| Stearic Acid | 0.010 |
| TOTAL | 0.500 |

Procedure:
1. Blend Chlorophytum species or Chlorophytum arundinaceum and Withania somnifera extracts (1:1) and purified Shilajit, Microcrystalline Cellulose, Croscarmellose Sodium and Syloid (screened through 30 mesh) in a suitable blender for 15 minutes.
2. Screen Stearic Acid through a 30 mesh, add to the above blender and mix for 5 minutes.
3. Compress into tablets with a target weight of 0.425 g.
4. Coat the tablets if necessary.

### Example 27

Tablets with anti-obesity, health-restorative and health promotional benefits dose: one tablet twice daily.

| Ingredient | Per Tablet, g |
|---|---|
| Chlorophytum species or Chlorophytum | 0.500 |
| arundinaceum and Withania somnifera | |
| extracts (1:1) | |
| Microcrystalline Cellulose | 0.150 |
| Syloid (Fumed Silicon Dioxide) | 0.005 |
| Croscarmellose Sodium | 0.010 |
| Stearic Acid | 0.010 |
| TOTAL | 0.675 |

Procedure:
1. Blend Chlorophytum species or Chlorophytum arundinaceum and Withania somnifera extracts (1:1), Microcrystalline Cellulose, Croscarmellose Sodium and Syloid (screened through 30 mesh) in a suitable blender for 15 minutes.
2. Screen Stearic Acid through a 30 mesh, add to the above blender and mix for 5 minutes.
3. Compress into tablets with a target weight of 0.675 g.
4. Coat the tablets if necessary.

### Example 28

Chewable tablets with anti-obesity, health-restorative and health promotional benefits: dose: 1-2 tablets twice a day.

| Ingredient | Per Tablet, g |
|---|---|
| Chlorophytum species or Chlorophytum | 0.379 |
| arundinaceum and Withania somnifera | |
| extracts (1:1), Taste-masked, 33% | |
| Sodium ascorbate | 0.098 |
| Microcrystalline Cellulose | 0.050 |
| Sodium Saccharin Powder | 0.002 |
| Compressible Sugar | 0.100 |
| Stearic Acid | 0.012 |
| Imitation Orange Flavor | 0.002 |
| FD&C Yellow #6 Dye | 0.001 |
| Fumed Silicon Dioxide (#30 mesh) | 0.006 |
| TOTAL | 0.650 |

Procedure:
1. Blend all the ingredients, except Stearic Acid, in a suitable blender for 15 minutes.
2. Screen steraic Acid thorugh a 30 mesh and blend with the above blend for 5 minutes.
3. Compress into tablets with a target weight of 0.650 g.

### Example 29

### Oral suspension with anti-obesity, health-restorative and health promotional benefits: dose: one teaspoonful twice a day.

| Ingredient | % |
|---|---|
| Chlorophytum species or Chlorophytum | 2.50 |
| arundinaceum and purified Shilajit (1:1) | |
| Colloidal magnesium aluminum silicate | 20.00 |
| premix (5% formula 21) | |
| Polaxamer 331 | 0.05 |
| Glycerin | 10.00 |
| Potassium sorbate | 0.20 |
| Sodium benzoate | 0.10 |
| Color | qs |
| Flavor | qs |
| Liquid sugar | 67.15 |
| Citric acid or Sodium hydroxide to pH 5.5 | qs |
| Purified water, qs | 100.0 |

Procedure:
1. Dissolve potassium sorbate, sodium benzoate and color in glycerin.
2. Add liquid sugar, colloidal magnesium aluminum silicate premix and half of the polaxamer 331 with agitation.
3. Disperse the rest of the polaxamer 331 and Chlorophytum species or Chlorophytum arundinaceum and purified Shilajit (1:1) with agitation.
4. Add flavor and pass the suspension through a colloid mill or a homogenizer rinsing thoroughly with purified water.
5. Adjust pH to 5.5 with either Citric acid or Sodium hydroxide solution.
6. Add purified water to make the final volume and mix well.
   Nutaceuticals

### Example 30

Maintenance B-Complex vitamin tablets or capsules with anti-obesity, health-restorative and health promotional benefits: dose: 1-2 tablets/capsules every day.

| Ingredient | Per Tablet/Capsule, mg |
|---|---|
| Chlorophytum species or Chlorophytum | 50.00 |
| arundinaceum and Withania somnifera | |
| extracts (1:1) | |
| Vitamin A acetate (dry from 500 IU) | 11.00 |
| Thiamini mononitrate, USP | 1.65 |
| Riboflavin, USP | 2.10 |
| Pyridoxine HCl, USP | 2.10 |
| Cyanocobalamine, 1% | 4.50 |
| D-Calcium pantothenate, USP | 7.50 |
| Niacinamide, USP | 22.00 |
| Dicalcium phosphate dihydrate, USP | 26.20 |
| Microcrystalline cellulose, NF | 61.95 |
| Talc, USP | 6.00 |
| Stearic acid, NF | 3.00 |
| Magnesium stearate, NF | 2.00 |
| TOTAL | 200.00 |

Procedure:
1. Blend all the ingredients, except Stearic acid and Magnesiun stearate, in a suitable blender for 15 minutes.
2. Add Stearic acid and Magnesium stearate and blend for 5 minutes.
3. Compress into 200 mg tablets or fill into capsules with a target fill weight of 200 mg.

What is claimed is:

1. A composition for the treatment, prevention or management of a weight related condition in primates comprising an effective amount of a plant extract of Chlorophytum species capable of reducing body weight.

2. The composition of claim 1 wherein the plant extracts is obtained from Chlorophytum arundinaceum.

3. The composition of claim 1 wherein the plant extracts is obtained from Chlorophytum borivilianum, C. tuberosum, C. malabenicum, C. attenuatum, C. breviscapum.

4. A formulation for the treatment, prevention or management of a condition in primates comprising:
an effective amount of a plant extract of Chlorophytum species capable of reducing body weight; and
pharmaceutically, nutritionally or veterinary acceptable excipients.

5. A formulation of claim 4 wherein the plant extracts is obtained from Chlorophytum borivilianum, C. tuberosum, C. malabenicum, C. attenuatum, C. breviscapum.

6. A method for reducing cholesterol, of a weight related condition comprising administering a composition comprising an effective amount of a plant extract.

7. The method of claim 6 wherein the weight related condition is obesity.

8. A pharmaceutical, nutritional or veterinary preparation of claim 6 is administered once or twice a day to a primate.

9. The formulation of claim 4 further comprising an adaptogen.

10. The formulation of claim 4 further comprising an antioxidant.

11. A formulation of claim 5 further comprising effective amounts of transition and/or trace metals.

12. The formulation of claim 11 wherein the transition or trace metals comprise of copper, chromium, zinc, selenium and/or metal ions ranging from 1 to about 500 ppm levels.

13. The formulation of claim 5 further comprising at least one additional anti-obesity ingredient other than the inventive composition.

14. The formulation of claim 13 wherein the additional anti-obesity ingredient comprises one or more of conjugated linoleic acid, bitter orange, hydroxycitric acid, chitosan, startch blockers, dehydroepiandesterone (DHEA), adaptogen, or mixtures thereof.

15. A method for the treatment, prevention or management of body weight in primates, comprising administering to a primate a formulation comprising:
a) a plant extract comprising an effective amount of spirosta-steroidal saponins comprising diosgenin, tigogenin, neotigogenin and sarsasapogenin as the genin components and mono-, di- and oligosaccharides, comprising glucose, rhamnose, arabinose, galactose and xylose as glycosidic components; and
b) a suitable excipient(s) allowed for pharmaceutical, nutritional and veterinary applications.

16. The method of claim 15 wherein the plant extract comprises an effective amount of spirosta-steroidal saponins obtained from Chlorophytum borivilianum, C. arundinaceum, C. tuberosum, C. malabenicum, C. attenuatum, C. breviscapum.

17. The method of claim 15 wherein the plant extract comprises an effective amount of spirosta-steroidal saponins obtained from Trigonella foenum-graecum L. (Papilionaceae), Digitalis purpurea L. (Scrophulariaceae), Dioscorea floribunda Mart & Gal. (Dioscoreaceae), Costus speciosus Sm. (Zingiberaceae), Asparagus racemosus Willd. (Liliaceae), Tribulus terrestris L. (Zygophyllaceae), or Solanum hispidum Pers. (Solanaceae).

18. A formulation for the treatment, prevention or management of body weight in primates, comprising:
a) spirosta-steroidal saponins comprising diosgenin, tigogenin, neotigogenin and sarsasapogenin as the major genin components and mono-, di- and oligosaccharides, comprising glucose, rhamnose, arabinose, galactose and xylose as glycosidic components;
b) spirosta-steroidal glycoalkaloids comprising solasodine and tomatidine as the alkaloidal aglycones, and mono-, di- and oligosaccharides, comprising glucose, rhamnose, arabinose, galactose and xylose as glycosidic components;
c) galacto-glucan oligosaccharides, and
d) suitable excipient(s) for pharmaceutical, nutritional and veterinary applications.

19. The formulation of claim 18 further comprising constituents belonging to phenolic dibenzyl.

20. The formulation of claim 18 wherein the spirosta-steroidal alkaloids is obtained from Chlorophytum borivilianum, C. arundinaceum, C. tuberosum, C. malabenicum, C. attenuatum, C. breviscapum.

21. The formulation of claim 18 wherein the spirosta-steroidal saponins is obtained from Trigonella foenum-graecum L. (Papilionaceae), Digitalis purpurea L. (Scrophulariaceae), Dioscorea floribunda Mart & Gal. (Dioscoreaceae), Costus speciosus Sm. (Zingiberaceae), Asparagus racemosus Willd. (Liliaceae), Tribulus terrestris L. (Zygophyllaceae), or Solanum hispidum Pers. (Solanaceae).

22. The formulation of claim 18 wherein the glacto-glucan oligosaccharides is obtained from Chlorophytum borivilianum, C. arundinaceum, C. tuberosum, C. malabenicum, C. attenuatum, C. breviscapum.

23. The formulation of claim 18 wherein the glacto-glucan oligosaccharides is obtained from Trigonella foenum-graecum L. (Papilionaceae), Digitalis purpurea L. (Scrophulariaceae), Dioscorea floribunda Mart & Gal. (Dioscoreaceae), Costus speciosus Sm. (Zingiberaceae), Asparagus racemosus Willd. (Liliaceae), Tribulus terrestris L. (Zygophyllaceae), or Solanum hispidum Pers. (Solanaceae).

24. The method of claim 15 wherein the formulation further comprises effective amounts of transition and/or trace metals.

25. The formulation of claim 18 wherein the formulation further comprises effective amounts of transition and/or trace metals.

26. The formulation of claim 25 wherein the transition or trace metals comprise of copper, chromium, zinc, selenium and/or metal ions ranging from 1 to about 500 ppm levels.

27. The method of claim 15 wherein the formulation further comprises at least one additional anti-obesity ingredient other than the inventive composition.

28. The method of claim 27 wherein the additional anti-obesity ingredients comprises at least one of conjugated linoleic acid, bitter orange, hydroxycitric acid, chitosan, startch blockers, dehydroepiandesterone (DHEA), adaptogen, or mixtures thereof.

29. The formulation of claim 19 wherein the formulation is combined with effective amounts of transition and/or trace metals or mixtures thereof.

30. The formulation of claim 29 wherein the transition or trace metals comprise of copper, chromium, zinc, selenium and/or metal ions ranging from 1 to about 500 ppm levels.

31. The formulation of claim 18 wherein the formulation further comprises at least one additional anti-obesity ingredient other than the inventive composition.

32. The formulation of claim 31 wherein the additional anti-obesity ingredients comprise at least one of conjugated linoleic acid, bitter orange, hydroxycitric acid, chitosan, startch blockers, dehydroepiandesterone (DHEA), adaptogen or mixtures thereof.

33. A method for reducing cholesterol, triglycerides, low density lipids and cortisol in primates comprising administering to a primate a formulation comprising
a) a plant extract comprising an effective amount of spirosta-steroidal saponins comprising diosgenin, tigogenin, neotigogenin and sarsasapogenin as the genin components and mono-, di- and oligosaccharides, comprising glucose, rhamnose, arabinose, galactose and xylose as glycosidic components and
b) a suitable excipient(s) allowed for pharmaceutical, nutritional and veterinary applications.

34. A method of claim 15 wherein the formulation is administered once or twice a day to a primate.

35. The method of claim 33 wherein the formulation further comprises at least one additional anti-obesity ingredient.

36. The formulation of claim 29 wherein the transition or trace metals comprise of copper, chromium, zinc, selenium and/or metal ions ranging from 1 to about 500 ppm levels.

37. An anti-obesity composition for primates, comprising of an effective amount of a plant extract of Chlorophytum species comprising of spirosta-steroidal saponins, spirosta-steroidal alkaloids and galacto-glucan oligosaccharides

38. The composition of claim 37 further comprising one or more additional constituents belonging to phenolic dibenzyl, having potent anti-oxidant and immuno-modulatory activities.

39. An anti-obesity composition for primates comprising an effective amount of a plant extract of Chlorophytum arundinaceum wherein the extract contains bioactive principles comprising spirosta-steroidal saponins, spirosta-steroidal alkaloids and galacto-glucan oligosaccharides.

40. The anti-obesity composition of claim 39, wherein the bioactive principles are isolated and characterized from the fresh tuber-roots of a cultivated variety of Chlorophytum arundinaceum.

41. The anti-obesity composition of claim 37 further comprising a pharmaceutically, nutritionally or veterinary acceptable excipients and effective amounts of antioxidant(s), adaptogen(s), transition and/or trace metals or mixtures thereof to form a formulation.

42. The formulation of claim 41 further comprising effective amounts of antioxidant(s), adaptogen(s), transition and/or trace metals.

43. The formulation of claim 42 wherein the antioxidant is obtained from Phyllanthus emblica, the adaptogen is obtained from Withania somnifera, and the transition metal is chromium complexed with Phyllanthus emblica extract.

44. The formulation of claim 18 further comprising one or more constituents from the group of antioxidants, adaptogens, vitamins, minerals or mixtures thereof.

45. The formulation of the composition of claim 41 wherein the adaptogen used is purified Shilajit or Withania somnifera or Panax ginseng or Siberian ginseng or mixture thereof.

46. The formulation of claim 18 further comprising one or more of the following constituents:
a) one or more amino acids comprising of alanine, glycine, valine, isoleucine, proline lysine, serine, threonine, aspartic acid, ornithine, glutamic acid, phenyl alanine, tyrosine, arginine.
b) one or more phytosterols, sitosterols and sterols belonging to chlolest-7-en-6-one, 3-(acetoxy)-9-hydroxy, cholesta-7,9(11)-dien-3-ol, 4,4-dimethyl, cholest-8(14)-en-3-one, cholest-8(14)-en-3-ol, ergosterol, androstan-17-one-3-hydroxy.
c) one or more reductone and related derivatives belonging to 2-ketogluconolactone, 2-ketogluconolactone-6-phosphate family of compounds.

47. The formulation of claim 46 further comprising effective amounts of transition and/or trace metals or mixtures thereof.

48. The formulation of claim 47 wherein the transition and/or trace metals or mixtures thereof are in the range of 1 to 500 ppm.

49. The formulation of claim 46 further comprising at least one additional anti-obesity ingredient other than the inventive composition.

50. The formulation of claim 49 wherein the additional anti-obesity ingredients comprises one or more of conjugated linoleic acid, bitter orange, hydroxycitric acid, chitosan, startch blockers, dehydroepiandesterone (DHEA), adaptogen, or mixtures thereof.

51. A formulation of claim 46 wherein the anti-obesity composition is combined with pharmaceutically, nutritionally or veterinary acceptable excipients and effective amounts of antioxidant(s), adaptogen(s), transition and/or trace metals or mixtures thereof.

52. The formulation of claim 51 wherein the formulation is further comprised of purified Shilajit or mixtures thereof.

53. The formulation of claim 51 wherein the antioxidant obtained from Phyllanthus species, adaptogen obtained from Withania somnifera and the transition metal is chromium complexed with Phyllanthus emblica extract.

54. The formulation of the composition of claim 53 wherein the antioxidant obtained from Phyllanthus emblica.

55. The formulation of claim 51 wherein the antioxidant obtained from Phyllanthus emblica, adaptogen obtained from purified Shilajit and the transition metal is chromium complexed with Phyllanthus emblica extract.

56. The formulation claim 51 wherein the adaptogen obtained from Withania somnifera or purified Shilajit or mixtures thereof.
